(19) United States
(12) Patent Application Publication
(10) Pub. No.: US 2010/0087628 A1
(43) Pub. Date: Apr. 8, 2010

(54) ISOLATION AND SEPARATION OF MINIMALLY DENATURED POTATO PROTEINS AND PEPTIDES

(75) Inventors: Allen Otto Fog Lihme, Birkerod (DK); Marie Bendix Hansen, Niva (DK); Martin Pontoppidan, Lyngby (DK)

Correspondence Address: FOLEY AND LARDNER LLP;SUITE 500, 3000 K STREET NW, WASHINGTON, DC, 20007, US

(73) Assignee: Upfront Chromatagraphy A/S

(21) Appl. No.: 12/524,817

(22) PCT Filed: Jan. 29, 2008
(86) PCT No.: PCT/DK08/00039

§ 371 Date: Oct. 5, 2009

(30) Foreign Application Priority Data
Jan. 29, 2007 (DK) PA 2007 00139

(51) Int. Cl. C07K 14/415; C07K 1/22

(52) U.S. Cl. 530/370; 530/413

(57) ABSTRACT

The present invention relates to the large-scale fractionation and isolation of peptides, polypeptides and protein(s) from a potato derived protein solution such as potato extract, potato fruit juice and fruit water using an adsorbent coupled with a ligand for the capture of the protein(s), from the protein solution. In particular the invention relates to a process for the isolation and separation of patatin and potato protease inhibitors using a low temperature non-denaturing process.

### FIELD OF THE INVENTION

The present invention relates to the large-scale fractionation and isolation of peptides, polypeptides and protein(s) from a potato derived protein solution such as potato extract, potato fruit juice and fruit water using an adsorbent coupled with a ligand for the capture of the protein(s), from the protein solution. In particular the invention relates to a process for the isolation and separation of patatin and potato protease inhibitors using a low temperature non-denaturing process.

### TECHNICAL BACKGROUND AND PRIOR ART

The potato belongs to the Solanaceae or nightshade family whose other members include tomatoes, eggplants, peppers, and tomatillos. They are the swollen portion of the underground stem which is called a tuber and is designed to provide food for the green leafy portion of the plant. If allowed to flower and fruit, the potato plant will bear an inedible fruit resembling a tomato.

The juice of the potato tubers is a waste, which gives environmental problems in the production of potato starch. One possible means of reducing this pollution problem is the recovery of proteins, which can represent up to 250 g/kg of the soluble dry solids of potato juice. In the professional vocabulary the undiluted juice from the potato tuber is called potato fruit juice, whereas the diluted juice is designated potato fruit water. Both have a high content of organic materials which give rise to high oxygen demand (high BOD and COD) in waste water from the potato starch plants. The potato fruit water also contains phosphorous- and nitrogen-compounds which fertilize the recipients. Concentrating potato fruit water to be used as feed supplement by evaporation or reverse osmosis is used by some potato starch manufacturers. Reverse osmosis, which is not as energy demanding as evaporation, does however demand that the potato fruit water is pretreated and filtered clear to avoid clogging of the membranes which hold inorganic salts and low molecular organic components back in the concentrate.

Fresh potato juice is a complex mixture of soluble and insoluble material comprising proteins, starch, minerals, toxic glycoalkaloides, and monomeric and polymeric reactive phenols. The oxidation of natural phenolic compounds in potato juice causes them to turn brown or black. Chemically, the phenolic compounds are oxidized into quinones, which rapidly combine into a dark polymer residue. During the oxidation process reaction and partial cross-linking of the proteins may occur very rapidly. Thus, from a technological point of view the complexity and instability of the potato juice makes the separation and isolation of minimally denatured or modified potato proteins much more complicated and economically demanding than the isolation of proteins from other types of protein solution.

Potato proteins can tentatively be divided into three classes (i) the patatin family, highly homologous acidic 43 kDa glycoproteins (40-50%), (ii) basic 22 kDa proteins family (30-40%) and (iii) basic protease inhibitors (20-30%). Patatin is a family of glycoproteins that have lipid acyl hydrolase and transferase activities and account for up to 40% of the total soluble protein in potato tubers.

Potato protein has traditionally been regarded as a waste product of starch manufacture. However, its nutritional qualities (i.e. protein efficiency ratio and biological value) have been shown to be greater than that of casein and comparable to that of whole egg. Potato protein is rich in lysine and theoretically an excellent supplement for lysine-poor proteins such as those of cereals. Despite its unique nutritional qualities, potato protein is currently only used as animal feed, because the available products exhibit a number of serious drawbacks.

One of the major drawbacks is that the recovery of potato protein from the effluent of potato starch mills is commonly carried out on an industrial scale by heat coagulation. Prior attempts to isolate the proteins from the potato juice by more mild methods, such as membrane filtration and precipitation have proven to be inefficient in the industrial scale production environment. Membrane filtration applied directly to unclarified and clarified potato juice has proven to be very complicated and inefficient due to heavy fouling of the membranes and concomitant loss of flux and separation ability. Both membrane filtration and precipitation methods have significant drawbacks when applied directly to the potato juice due to the lack of selectivity between the desired protein product and other components in the raw material. Membrane filtration, for example, cannot separate the high molecular weight protein product from polymerised phenolic compounds or polysaccharides since the membrane will tend to retain them all.

Due to the presently applied heat coagulation processes, potato protein becomes heavily denatured and as a consequence becomes devoid of functional properties, i.e. emulsifying capacity, foaming capacity, thermo-gelling capacity, water binding capacity. Even the most essential requirement for its application in the food industry, i.e. solubility in water, cannot be met.

This invention relates to methods for the isolation of native potato proteins, polypeptides and peptides with minimal denaturation, high purity and minimal production costs. Minimally denatured potato protein may be used as a substitute for animal protein, fat or hydrocolloids in food compositions. This allows for a diet based on vegetable proteins, with high nutritional value.

The potato protein is preferably isolated from potato fruit juice, a waste product of the starch manufacturing industry. Both diluted and undiluted potato fruit juice may be used. Other suitable sources of undenatured potato protein include for example potato peel extracts and effluent streams from potato processing industries other than the potato-starch industry.

In this context, minimally denatured potato protein refers to potato protein, which has retained most of its intrinsic functional properties, such as emulsifying capacity, solubility, foaming capacity, water binding capacity and thermo-gelling capacity, on isolation. As a consequence, its functional properties will be better than those of denatured potato protein produced by e.g. heat coagulation and at least as good as those of soy protein.

Patatins are a family of proteins found in potato and other plants, particularly in solanaceous plants. In potato, the patatins are found predominantly in tubers, but also at much lower levels in other plant organs.

It has been discovered that patatins, the major storage protein of potato tubers, will control various insects, including western corn rootworm (WCRW), Diabrotica virgifera, southern corn rootworm (SCRW), Diabrotica undecimpunctata, and boll weevil (BWV), Anthonomus grandis. Patatins are lethal to some larvae and will stunt the growth of survivors so that maturation is prevented or severely delayed resulting in no reproduction. These proteins, which are known to have esterase (lipid acyl hydrolase) activity, may be applied directly to plants and thereby exhibit insect control.

Proteins that inhibit proteolytic enzymes are often found in high concentrations in many seeds and other plant storage organs. Inhibitor proteins are also found in virtually all animal tissues and fluids. These proteins have been the object of considerable research for many years because of their ability to complex with and inhibit proteolytic enzymes from animals and microorganisms. The inhibitors have become valuable tools for the study of proteolysis in medicine and biology. Protease inhibitors are of particular interest due to their therapeutic potentials in controlling proteinases involved in a number of disorders such as pancreatitis, shock, and emphysema, and as agents for the regulation of mammalian fertilization. Potato tubers are a rich source of a complex group of proteins and polypeptides that potently inhibit several proteolytic enzymes usually found in animals and microorganisms. In particular, potato inhibitors are known to inhibit human digestive proteinases, and thus have application in the control of obesity and diabetes.

Many of these protease inhibitors inhibit the activity of digestive proteinases, such as trypsin and chymotrypsin that naturally occur in both insects and mammals. By disturbing the natural digestive process, proteinase inhibitors form part of a plant's natural defense against foraging by herbivores. For the same reason proteinase inhibitors have application in the pest control industry for the control of insects such as fire ants.

Two broad classes of protease inhibitor super-families have been identified from soybean and other legumes with each class having several iso-inhibitors. Kunitz-type inhibitor is the major member of the first class whose members have 170-200 amino acids, molecular weights between 20,000 and 25,000, and act principally against trypsin. Kunitz-type proteinase inhibitors are mostly single chain polypeptides with 4 cysteines linked in two disulfide bridges, and with one reactive site located in a loop defined by disulfide bridge. The second class of inhibitors contains 60-85 amino acids, has a range in molecular weight of 6000-10,000, has high proportion of disulfide bonds, is relatively heat-stable, and inhibits both trypsin and chemotrypsin at independent binding sites. Bowman-Birk inhibitor is an example of this class. Kunitz inhibitor is capable of inhibiting trypsin derived from a number of animal species as well as bovine chemotrypsin, human plasmin, and plasma kallikrein. The cationic form of human trypsin, which accounts for a majority of trypsin activity, is only weakly inhibited by the Kunitz inhibitor, whereas the anionic form is fully inhibited. The Bowman-Birk inhibitor is a 71 amino acid chain protein with 7 disulfide bonds characterized by its low molecular weight of about 8000 (in non-associated monomers), high concentration (about 20%) of cystine, high solubility, resistance to heat denaturation and having the capacity to inhibit trypsin and chymotrypsin at independent inhibitorysites. The major effects of proteinase inhibitors in animal diets include growth depression and pancreatic hypertrophy. Resistance of raw soybean protein to proteolysis, low levels of sulfur-containing amino acids in soybean proteins, and lower digestibility, absorption, and utilization of available nitrogen from the small intestine due to the presence of proteinase inhibitors, all appear to contribute to growth depression.

Proteinase inhibitors extracted from potatoes have been distinguished into two groups based on their heat stability. The group of inhibitors that is stable at 80° C. for 10 minutes have been identified as inhibitor I (mol. wt. 39,000), carboxypeptidase inhibitor (CPI) (mol. wt. 4,100), inhibitors IIa and IIb (mol. wt. 20,700) and inhibitor A5 (mol. wt. 26,000).

In addition to inhibitor I and inhibitor II, several low molecular weight inhibitors have been detected in potato. Among them are the carboxypeptidase inhibitor, which has been extensively characterized and at least three inhibitors of serine proteinases. The amino acid sequences of two low molecular weight serine proteinase inhibitors from Russet Burbank potatoes have been determined. One of those, a chemotrypsin inhibitor, is a peptide of 52 amino acid residues, while the second inhibitor, which is specific for trypsin, contains 51 amino acid residues. These peptides are highly homologous, differing at only nine positions. At position 38, the chymotrypsin inhibitor possesses leucine and the trypsine inhibitor an arginine. The inhibitors are also homologous with potato inhibitor II and with an inhibitor previously isolated from eggplants (Hass, et al., 1982).

Protease inhibitors from potato have a broad range of potentially important applications. U.S. Pat. No. 5,187,154 describe a method for the diagnosis and the treatment of individuals with diabetes or at risk to develop diabetes mellitus. In particular, gastric emptying determinations are used to assess risk. Risk or early symptoms associated with subsequent development of diabetes mellitus may be controlled or alleviated by delaying gastric emptying, which was achieved by the administration of cholecystokinin.

U.S. Pat. No. 4,491,578 describe a method of eliciting satiety in mammals through the administration of an effective amount of a trypsin inhibitor. The method was based on the postulate that the enzyme trypsin, normally secreted by the pancreas, constitutes a negative feedback signal for cholecystokinin secretion that in turn comprises a putative satiety signal. Thus, the effect of the trypsin inhibitor is to increase the concentration of cholecystokinin secretion advancing the sensation of satiety resulting in a consequent decrease in food intake and, overtime, body weight.

U.S. Pat. No. 4,906,457 describe compositions and methods for reducing the risk of skin cancer. The described compositions included at least one effective protease inhibitor. Preferred protease inhibitors included serine protease inhibitors and metallo-protease inhibitors. The protease inhibitors were preferably included in concentrations ranging from approximately 10 picograms to 10 milligrams per milliliter of the skin-applicable topical mixtures. The topical mixtures preferably included a suitable topical vehicle such as a cream, lotion, or ointment. One class of anti-carcinogenic skin treatment compositions of this invention preferably included the desired protease inhibitors in combination with a suitable sunscreen agent or agents, such as para-amino benzoic acid, to provide particularly advantageous compositions for reducing the risk of sunlight-induced skin cancer.

Protease inhibitors preserve raw materials of animal origin and materials which contain or are contaminated by proteases, by inhibiting the hydrolysis of proteins to amino acids. In this way the proteins are made unavailable to the digestion system of the microorganisms. Owing to a high content of proteases, prey-containing fish gives reduction in quality as well as difficulties in processing when used as raw material for the fish-meal and fish-oil industry. In laboratory experiments and in whole scale tests under actual fishing, it has been found that protease inhibitors extracted from potato juice efficiently hinder the dissolving of proteins and the formation of biogenic amines in prey-containing fish for industrial use. Potato-inhibitors from the juice of the potato make prey-containing fish keep better in storage. For the fishmeal industry this means that problematic raw material of low storageability may be upgraded to production of fishmeal of a better quality than meal produced from fish without this protease inhibitor treatment.

According to this invention, one type of relevant raw material for the isolation and separation of proteins and protease inhibitors is the fruit juice from the production of potato starch. Small scale extraction of proteins and protease inhibitors from potatoes is described in the literature. The dry matter content of the potatoes varies depending on the potato variety and growth conditions between 20 and 30 g dry matter/100 g potato tuber. The dry matter of the potato tubers contains considerable quantities of protein, 5-6 g 20 protein/100 g dry matter. As a mean, the potato tuber contains ¾ water and ¼ dry matter. The starch and the fibers occur as particulate material, and make up the larger part of the dry matter. The liquid phase has varying content of dissolved dry matter, and the Patatin and protease inhibitors make up part of this dissolved dry matter. The starch is available as granules suspended in the potato juice, which again is enclosed in cells. The extraction of the starch starts by grinding the potato tubers in such a way that the cell walls are broken and the juice with starch granules and fibers is made free and make a slurry. The starch granules and the fibers are afterwards mechanically separated from the potato fruit juice. The juice contains phenol compounds, which on oxidation give coloured compounds. Most noticeable is the oxidation of tyrosin to melamin and further to compounds with brown and black colour. Addition of sulphite as sulphurdioxidegas (SO₂.gas) or sulphites gives a reducing environment, which prevents the formation of coloured compounds, and at the same time sulphite protects the protease inhibitors and other proteins against damage. In some potato starch factories the potato tubers are fine ground with high speed grating machines, and the starch granules and the fibers separated. The process typically makes use of rotating conical sieves, nozzle separators and vacuum filters. The potato fruit juice may be recirculated and used for transporting the slurry and to wash the starch granules through the fine gauze of the sieves.

Sulphur dioxide gas is often added to the potato slurry to prevent oxidation. Normally about 0.2 kg sulphur dioxide (SO₂ gas)/ton potatoes is used. Undiluted potato fruit juice may also be separated from the finely ground potato-mass by the use of decanter centrifuges. Decanter centrifuges are centrifuges with conical, rotating drums where heavy solids move to the periphery and settle to the drum surface and are continuously transported out by means of a screw and leaves the centrifuge. The potato fruitjuice leaves the decanter centrifuge through an adjustable overflow. The solid material is mostly made up of starch and fibers. The starch may be separated from the fibers and worked up into potato starch, or the mixed solid material from the decanter centrifuge may be used as raw material in potato distilleries. The potato fruit juice separated by both these methods may be used in the production of protease inhibitors, other proteins and especially patatin by the methods described in this patent application. By using the invention, the environmental problems previously stated are reduced for the reason that part of the proteins leaves the plant as product.

U.S. Pat. No. 6,042,872 relates to a method for preparing purified heat-coagulated potato protein, wherein heat-coagulated potato protein, after being separated from potato juice, is treated with one or more aqueous solutions of one or more inorganic acids, and thereafter is recovered. The treatment is preferably carried out at a pH between 1 and 5. The invention further relates to animal feed compositions, which contain a prepared purified heat-coagulated potato protein as a component.

Accordingly, a process for the isolation of minimally denatured potato proteins and peptides which is fast, robust (i.e. being reliable during daily operation with low down time), specific and safe, and which at the same time provides a yield and purity of the products of interest during processing and thereby facilitates an improved and acceptable balance between yield and economy, compared to the conventionally used processes, e.g. the processes based on heat coagulation, and which solves the above mentioned problems is therefore desired. Such a process is provided herein.

### SUMMARY OF THE INVENTION

The present invention relates to a process for the isolation and/or fractionation of peptide, polypeptide or protein from potato juice. The process of the present invention is fast, robust, specific and safe, and provides an improved yield and purity of the product of interest during processing and thereby facilitates an improved and acceptable balance between yield of product and economy involved, compared to the conventionally used methods. The process according to the invention is particularly suitable for large-scale production of minimally denatured protein products.

### DETAILED DISCLOSURE OF THE INVENTION

Thus, in one aspect the present invention provides a process for the isolation of one or more protein(s) or peptides from a protein solution wherein the protein is obtained from a potato derived source, said process comprising the steps of:
a) optionally adjusting the pH of the protein solution to a preset pH;
b) optionally adjusting the ionic strength or conductivity of the protein solution to a preset ionic strength or a preset conductivity;
c) applying said protein solution to an adsorption column comprising an adsorbent, wherein the adsorbent comprises a functionalized matrix polymer carrying a plurality of covalently attached functional groups comprising an aromatic or heteroaromatic ring-system and one or more acidic groups
e) optionally washing the column;
f) obtaining the one or more protein(s) from the adsorbent.

In a further aspect the present invention provides a process for the isolation of one or more protein(s) from a protein solution wherein the protein is selected from the group consisting of Patatin(s) and vegetable derived protease inhibitors, said process comprising the steps of:
a) optionally adjusting the pH of the protein solution to a preset pH;
b) optionally adjusting the ionic strength or conductivity of the protein solution to a preset ionic strength or a preset conductivity;
c) applying said protein solution to an adsorption column comprising an adsorbent, wherein the adsorbent comprises a functionalized matrix polymer carrying a plurality of covalently attached functional groups comprising an aromatic or heteroaromatic ring-system and one or more acidic groups;
d) optionally washing the column;
e) obtaining the one or more protein(s) from the adsorbent.

In a further aspect the present invention provides a process for the isolation of one or more protein(s) from a protein solution wherein the protein is selected from the group consisting of Patatin(s) and vegetable derived protease inhibitors, said process comprising the steps of:
a) optionally adjusting the pH of the protein solution to a preset pH;
b) optionally adjusting the ionic strength or conductivity of the protein solution to a preset ionic strength or a preset conductivity;
c) applying said protein solution to an adsorption column comprising an adsorbent, wherein the adsorbent has a density above 1.5 g/ml;
d) optionally washing the column;
e) obtaining the one or more protein(s) from the adsorbent.

In this context the term “potato juice” shall mean any type of liquid stream obtained from the industrial production of potato starch, whether it is the undiluted juice from the potato tuber also called potato fruit-juice or a diluted juice (also designated potato fruit-water) optionally mixed with washing water.

In this context the term “crude or briefly pre-treated potato juice” shall mean that the potato juice is practically as obtained from the starch process except for any brief and non-denaturing pre-treatments such as pH-adjustment, clarification and or colour removal (e.g. by centrifugation, filtration, flocculation or adsorption) that does not significantly alter the content and nature of the Patatin and the protease inhibitors in the potato juice.

The Protein Solution and its Source

In accordance with the present invention the protein(s) and peptides of interest may be separated and isolated from a protein solution. In the present context the term “protein solution” relates to any kind of solution in liquid form comprising the protein(s) or peptides of interest and from which the protein(s) may be separated and isolated. In an embodiment of the present invention the protein solution may be obtained from extracts or juice derived from plants belonging to the Solanum genus such as the Potato family (Nightshade family)—Solanaceae—which comprises about 2500 species of plants spread all over the world. They are herbs, trees and shrubs. Many of these species are very important for mankind because of their value as food (potatoes, tomatoes, peppers, etc.).

In an embodiment of the present invention the plant selected may be capable of producing or may have been modified to produce the protein(s) and/or peptides of interest. This may be a transgenic plant, a plant part, or plant cell preferably which has been transformed with a nucleotide sequence encoding a protein or peptide of interest so as to express and produce the protein or peptide in recoverable quantities. Examples of relevant plant parts are stem, callus, leaves, root, fruits, seeds, and tubers as well as the individual tissues comprising these parts, e.g., epidermis, mesophyll, parenchyme, vascular tissues, meristems. Specific plant cell compartments, such as chloroplasts, apoplasts, mitochondria, vacuoles, peroxisomes and cytoplasm are also considered to be a plant part. Furthermore, any plant cell, whatever the tissue origin, is considered to be a plant part. Likewise, plant parts such as specific tissues and cells isolated to facilitate the utilisation of the invention are also considered plant parts, e.g., embryos, endosperms, aleurone and seeds coats.

The transgenic plant or plant cell expressing a protein to be isolated according to the invention may be constructed in accordance with methods known in the art. In short, the plant or plant cell is constructed by incorporating one or more expression constructs encoding a protein of interest into the plant host genome and propagating the resulting modified plant or plant cell into a transgenic plant or plant cell.

In the present context the term “supernatant” relates to a liquid phase, which is lying above a liquid fraction, a sediment fraction or a precipitated fraction obtained by the addition of an alcohol to the protein solution, in accordance with the present invention.

In the present context the term “fraction” relates to a portion of the protein solution, which may be separated from the supernatant by a fractionation process, such as filtration, micro-filtration, centrifugation, distillation or chromatography and the fraction may be either a combination of compounds or a pure compound.

In an embodiment of the present invention the temperature of the protein solution may be in the range of −5 to 50° C., more preferably in the range of −5 to 40° C., still more preferably in the range of −5 to 30° C., still more preferably in the range of −5 to 20° C., still more preferably in the range from 0 to 10° C. or in the range of 0 to 50° C., more preferably in the range of 10 to 50° C., still more preferably in the range of 15 to 50° C., still more preferably in the range of 18 to 50° C., still more preferably in the range from 30 to 50° C.

Adsorption

The Adsorbent

It is a further object of this invention to provide a process for isolation of protein(s) from a protein solution based on adsorption to any type of solid phase material of any shape and format including packed bed adsorption, batch adsorption, suspended bed adsorption, Expanded bed adsorption (EBA), fluidised bed adsorption and membrane based adsorption. Furthermore, the adsorption may be characterised by the use of selective adsorbent characteristics and/or ligand chemistry enabling the specific binding and subsequent elution of substantially only one substance, or alternatively enabling a group specific binding of a few substances followed by selective and consecutive elution of one or more substances from the adsorbent.

The adsorbent comprises a ligand suitable for binding to the one or more protein(s) and peptides of interest. In an embodiment of the present invention the adsorbent may optionally be washed and/or equilibrated with one or more washing buffer and/or equilibration buffers.

For a broad range of preferred embodiments of the present invention it may be of critical importance that the adsorbent is a particle having combined characteristics in terms of size and density. It has thus been found that for highly concentrated protein solutions such as potato juice it may be desirable to employ particles having a volume mean particle diameter of less than 250 μm in order to obtain a fast and efficient protein-binding (which is important for the productivity and thus the economy of a production plant). However it has further been found that it is the combination of the small diameter of the adsorbent particles (below 250 μm) with a certain minimum density (more than 1.2 g/ml)) of the adsorbent particles that enables significant improvements in production plant productivity. Hereby a unique combination of fast and efficient protein binding with high liquid flow rates through the columns employed for the adsorption process may be achieved. Particularly for non-packed columns such as e.g. expanded bed columns and suspended bed columns the high liquid flow rates obtainable with the adsorbents according to the invention may be significant. For packed bed columns it may be a distinct advantage that the small adsorbent particles have a high density providing fast sedimentation during the packing and re-packing procedure, which otherwise is a slow and demanding process step.

Generally it is found that a smaller mean volume diameter of the particles may desire a higher density of the particles.

Examples of commercial adsorbent particles that may be employed for some of the embodiments of the present invention are:

FastLine UFC NNSDW, UpFront Chromatography A/S, Denmark having a volume mean particles diameter of 70 μm and a density of 2.9 g/ml.

STREAMLINE SP, Amersham Biosciences, Sweden, having a volume mean particle diameter of 200 μm and a density of 1.2 g/ml.

STREAMLINE Direct CST-1, Amersham Biosciences, Sweden having a volume mean particle diameter of 135 μm and a density of 1.8 g/ml.

Q and CM HyperZ Ion exchange sorbents, Biosepra SA, France, having a volume mean particle diameter of 75 μm and a density of 3.2 g/ml.

Specifically in expanded bed adsorption the flow rate, the size of the particles and the density of the particles may all have influence on the expansion of the expanded bed and it is important to control the degree of expansion in such a way to keep the particles inside the column when working with Expanded bed adsorption. For industrial applicability it may be of interest to have a high flow-rate and a low expansion. The degree of expansion may be determined as H/H0, where H0 is the height of the bed in packed bed mode (without flow through the column) and H is the height of the bed in expanded mode (with a given flow through the column). In an embodiment of the present invention the degree of expansion H/H0 is in the range of 1.1-6, such as 1.1-5, e.g. 1.1-4, such as 1.2-5, e.g. 1.5-4 such as 2-4, such as, such as 2-3, such as 3-4. In another embodiment of the present invention the degree of expansion H/H0 is at most 1.2, e.g. at the most 1.3, such as at most 1.5, e.g. at most 1.8 such as at most 2, such as at most 2.5, e.g. at most 3, such as at most 3.5, e.g. at 4, such as at most 4.5. In yet an embodiment of the present invention the flow-rate is 5 cm/min and the degree of expansion H/H0 is at most 1.2, e.g. at the most 1.3, such as at most 1.5, e.g. at most 1.8 such as at most 2, such as at most 2.5, e.g. at most 3, such as at most 3.5, e.g. at 4, such as at most 4.5. In a further embodiment of the present invention the flow-rate is 7 cm/min and the degree of expansion H/H0 is at most 1.2, e.g. at the most 1.3, such as at most 1.5, e.g. at most 1.8 such as at most 2, such as at most 2.5, e.g. at most 3, such as at most 3.5, e.g. at 4, such as at most 4.5. In still an embodiment of the present invention the flow-rate is 10 cm/min and the degree of expansion H/H0 is at most 1.2, e.g. at the most 1.3, such as at most 1.5, e.g. at most 1.8 such as at most 2, such as at most 2.5, e.g. at most 3, such as at most 3.5, e.g. at 4, such as at most 4.5. In yet an embodiment of the present invention the flow-rate is 15 cm/min and the degree of expansion H/H0 is at most 1.2, e.g. at the most 1.3, such as at most 1.5, e.g. at most 1.8 such as at most 2, such as at most 2.5, e.g. at most 3, such as at most 3.5, e.g. at 4, such as at most 4.5. In still an embodiment of the present invention the flow-rate is 20 cm/min and the degree of expansion H/H0 is at most 1.2, e.g. at the most 1.3, such as at most 1.5, e.g. at most 1.8 such as at most 2, such as at most 2.5, e.g. at most 3, such as at most 3.5, e.g. at 4, such as at most 4.5.

In an embodiment of the present invention the linear flow rate of the packed bed column or the expanded bed column may be at least 2 cm/min, more preferably at least 3 cm/min, still more preferably at least 4 cm/min, still more preferably at least 5 cm/min, still more preferably at least 6 cm/min, still more preferably at least 7 cm/min, still more preferably at least 8 cm/min, still more preferably at least 10 cm/min, still more preferably at least 12 cm/min, still more preferably at least 15 cm/min, still more preferably at least 20 cm/min, still more preferably at least 25 cm/min, still more preferably at least 30 cm/min, still more preferably at least 40 cm/min, still more preferably at least 50 cm/min. In yet an embodiment of the present invention the linear flow rate is in the range of 1-75 cm/min, such as 2-75 cm/min, e.g. 5-75 cm/min, such as 7-75 cm/min, e.g. 10-75 cm/min, such as 15-75 cm/min, e.g. 20-75 cm/min, such as 30-75 cm/min, e.g. 40-75 cm/min, such as 50-75 cm/min, e.g. 1-50 cm/min, such as 2-50 cm/min, e.g. 2-30 cm/min, such as 3-30 cm/min, such as 3-20 cm/min, such as 3-15 cm/min, such as 4-30 cm/min, such as 4-25 cm/min, such as 4-20 cm/min, such as 4-15 cm/min, such as 5-25 cm/min, e.g. 5-15 cm/min, such as 5-10 cm/min, e.g. 5-7.5 cm/min, such as 7.5 cm/min. These increased flow rates, compared to conventional used flow rates (in particular for packed bed columns), may be possible to a great extent due to the small particle diameter in combination with the high density of the adsorbent.

In a particular embodiments of the present invention the application of protein solution to the adsorbent column may be performed with a linear flow rate of at least 200 cm/hour, such as at least 300 cm/hour, more preferably at least 400 cm/hour, such as at least 500 or 600 cm/hour, such as at least 900 cm/hour.

In an embodiment of the present invention the column may comprise a high-density adsorbent. In the present context the term “high-density adsorbent” relates to part of the group of adsorbents and involves the entire bed of adsorbent particles present in the adsorbent column. The term “adsorbent particle” is used interchangeably with the term “particle” and relates to the individual single particles which make up the adsorbent in the column. The preferred shape of a single adsorbent particle is substantially spherical. The overall shape of the particles is, however, normally not extremely critical, thus, the particles can have other types of rounded shapes, e.g. ellipsoid, droplet and bean forms. However, for certain applications (e.g. when the particles are used in a fluidised bed set-up), it may be preferred that at least 95% of the particles are substantially spherical. In the present context the terms “particle diameter” and “particle size” are used interchangeable and relates to the diameter of a circle which may be made around the particle and therefore, may be regarded as the diameter of the particle on the widest part of the particle.

The density of an adsorbent particle is meant to describe the density of the adsorbent particle in its fully solvated (e.g. hydrated) state as opposed to the density of a dried adsorbent. In the present invention the density of the particle may be measured by performing the following procedure: 1) Draining a sample of the adsorbent particles by gentle suction on a vacuum glass filter to remove the interstitial water occupying the space between the individual beads. 2) Weighing the drained particle sample to determine the total mass of the particles. 3) Adding the entire amount of drained particle sample to a known amount of water in a measuring cylinder and reading out the increase in total volume obtained by the addition of the drained particles. 4) Calculating the density by dividing the total mass of the drained particles with the volume increase determined under Item 3.

In an embodiment of the present invention the density of the adsorbent particle may be in the range of 1.2 g/ml to 20 g/ml, more preferably in the range of 1.5 g/ml to 20 g/ml, more preferably in the range from 1.9-20, more preferably in the range from 2.0 g/ml to 20 g/ml, more preferably in the range from 2.1 g/ml to 20 g/ml, more preferably in the range from 2.3 g/ml to 20 g/ml, even more preferably in the range of 2.5 g/ml to 20 g/ml, even more preferably in the range of 2.8 g/ml to 20 g/ml, e.g. in the range of 2.9 g/ml to 20 g/ml, still more preferably in the range of 3.0 g/ml to 20 g/ml, still more preferably in the range of 3.5 g/ml to 20 g/ml, still more preferably in the range of 4 g/ml to 20 g/ml, still more preferably in the range of 5 g/ml to 20 g/ml, still more preferably in the range of 10 g/ml to 20 g/ml, still more preferably in the range of 15 g/ml to 20 g/ml, still more preferably in the range of 4 g/ml to 15 g/ml, still more preferably in the range of 4 g/ml to 10 g/ml, still more preferably in the range of 1.5 g/ml to 15 g/ml.

The density of the EBA adsorbent particle may be significant for the applicable flow rates in relation to the maximal degree of expansion of the adsorbent bed possible inside a typical EBA column (e.g. H/H0 max 3-5) and may be at least 1.3 g/mL, more preferably at least 1.5 g/mL, still more preferably at least 1.8 g/mL, still more preferably at least 1.9 g/mL, even more preferably at least 2.0 g/mL, still more preferably at least 2.1 g/mL, most preferably at least 2.3 g/mL, even more preferably at least 2.5 g/ml, even more preferably at least 2.8 g/ml, even more preferably at least 2.9 g/ml, still more preferably at least 3.0 g/ml, still more preferably at least 3.5 g/ml in order to enable a high productivity of the process.

In yet an embodiment of the present invention 85% by volume of the individual particles of the adsorbent have a diameter within the range of 5 to 300 micron (μm), more preferably within the range of 30 to 300 micron, still more preferably within the range of 30 to 250 micron, still more preferably within the range of 30 to 200 micron, still more preferably within the range of 30 to 175 micron, still more preferably within the range of 50 to 175 micron, and even still more preferably within the range of 50 to 150 micron. In yet an embodiment of the present invention the mean particle diameter of the adsorbent may be 250 micron or less, preferably 200 micron or less, even more preferably 150 micron or less, still more preferably 125 micron or less, still more preferably 110 micron or less, still more preferably 100 micron or less, still more preferably 90 micron or less, still more preferably 80 micron or less.

Several parameters having an influence on the flow rate can be implemented in an EBA process. The fluidisation properties of the adsorbent particles (which may be described by the aid of Stokes Law) determine which flow rates that may be applied in order to expand the adsorbent and still keep the adsorbent inside the column. The main factors influencing this are the diameter and the density of the adsorbent particles in combination with the viscosity of the liquid flowing through the column. However, the binding and mass transfer kinetics relevant to a specific application are equally important to ensure optimal efficiency and productivity of the EBA process. For example, it may be possible to run an EBA column containing a certain EBA adsorbent at very high flow rates in terms of the physical fluidisation and expansion properties, while the applied high flow rate results in a poor and inefficient adsorption (i.e. a low dynamic capacity) due to the fact that the target molecules to be bound cannot diffuse in and out of the adsorbent particles to match this flow rate (i.e. the mass transfer kinetics is the limiting factor).

Consequently, in a combination of particularly preferred embodiments of the invention, where the applied linear flow rate during application of the protein solution is above 300 cm/hour, the mean volume particle diameter is 250 μm or less. Typically, in embodiments where the fractionation process is performed at an applied linear flow rate of above 500 cm/min, the mean volume particle diameter is below 200 μm, preferably below 150 μm. Typically, in embodiments where the fractionation process is performed at an applied linear flow rate of above 600 cm/hour, the mean volume particle diameter is preferably below 150 μm, more preferably below 120 μm.

Fundamentally the expression of particle size distribution in this context is volume based on the general understanding in the technical field and as described by Malvern Instruments Ltd (Worcestershire, UK) in their Operators guide (MAN 0320 Issue 1.0 March 2004) to the Mastersizer 2000E, which describes the measurement of particle size distribution by the aid of light scattering.

This means that, when the result indicates, for example, that 11% of a distribution is in the size category 65-78 μm, this means that the total volume of all particles with diameters in that range (within the size category 65-78) represents 11% of the total volume of all the particles in the distribution. The mean volume diameter (or volume mean diameter) referred to in the present context relates to the volume mean diameter labelled “D(4,3)” by Malvern for the Mastersizer 2000E. Whenever a particle size range is referred to such as “the particles have a particle diameter in the range of X-Y μm” it is meant to be understood as at least 90% of the total volume of particles have a diameter in the range of X-Y μm, such as at least 95%, e.g. at least 98%, such as at least 99%.

In still an embodiment of the present invention the adsorbent density, particle diameter and the mean volume particle diameter as described above may be combined in any way possible to provide the most suitable adsorbent for the isolation of the one or more protein(s) of interest. In an embodiment of the present invention the density of the adsorbent may be in the range of 1.5 to 10.0, 85% by volume of the individual particles of the adsorbent may have a diameter within the range of 10 to 250 micron, and the mean volume particle diameter may be in the range of 15 to 250 micron. In another embodiment of the present invention the density of the adsorbent may be in the range of 2.0 to 5.0, 85% by volume of the individual particles of the adsorbent may have a diameter within the range of 20 to 200 micron, and the mean volume particle diameter may be in the range of 75 to 150 micron. In yet an embodiment of the present invention the density of the adsorbent may be in the range of 2.5 to 3.5, 85% by volume of the individual particles of the adsorbent may have a diameter within the range of 50 to 180 micron, and the mean volume particle diameter may be in the range of 80 to 140 micron.

In a combination of preferred embodiments, where the mean volume particle diameter may be 200 μm or less, the particle density is at least 1.6 g/mL, more preferably at least 1.9 g/mL. When the mean volume particle diameter is less than 175 μm the density must be at least 1.8 g/mL or more preferable at least 2.0 g/mL, even more preferably at least 2.3 g/ml. When the mean volume particle diameter is less than 150 μm the density must be at least 2.0 g/mL, more preferable at least 2.3 g/mL and most preferable at least 2.5 g/mL.

In en embodiment of the present invention the adsorbent particle comprises a particle having a mean volume particle diameter of at the most 250 μm and a particle density of at least 1.5 g/ml; such as a particle density of at least 1.6 g/ml; e.g. a particle density of at least 1.9 g/ml; such as a particle density of at least 2.0 g/ml; e.g. a particle density of at least 2.3 g/ml; such as a particle density of at least 2.5 g/ml; e.g. a particle density of at least 2.8; e.g. a particle density of at least 3.0 g/ml; such as a particle density of at least 3.5 g/ml; e.g. a particle density of at least 4.0 g/ml; such as a particle density of at least 4.5 g/ml. Preferably, the adsorbent particle comprises a particle having a mean volume particle diameter of at the most 200 μm and a particle density of at least 1.5 g/ml; such as a particle density of at least 1.6 g/ml; e.g. a particle density of at least 1.9 g/ml; such as a particle density of at least 2.0 g/ml; e.g. a particle density of at least 2.3 g/ml; such as a particle density of at least 2.5 g/ml; e.g. a particle density of at least 2.8; e.g. a particle density of at least 3.0 g/ml; such as a particle density of at least 3.5 g/ml; e.g. a particle density of at least 4.0 g/ml; such as a particle density of at least 4.5 g/ml. More preferably, the adsorbent particle comprises a particle having a mean volume particle diameter of at the most 175 μm and a particle density of at least 1.5 g/ml; such as a particle density of at least 1.6 g/ml; e.g. a particle density of at least 1.9 g/ml; such as a particle density of at least 2.0 g/ml; e.g. a particle density of at least 2.3 g/ml; such as a particle density of at least 2.5 g/ml; e.g. a particle density of at least 2.8; e.g. a particle density of at least 3.0 g/ml; such as a particle density of at least 3.5 g/ml; e.g. a particle density of at least 4.0 g/ml; such as a particle density of at least 4.5 g/ml. Even more preferably, the adsorbent particle comprises a particle having a mean volume particle diameter of at the most 150 μm and a particle density of at least 1.5 g/ml; such as a particle density of at least 1.6 g/ml; e.g. a particle density of at least 1.9 g/ml; such as a particle density of at least 2.0 g/ml; e.g. a particle density of at least 2.3 g/ml; such as a particle density of at least 2.5 g/ml; e.g. a particle density of at least 2.8; e.g. a particle density of at least 3.0 g/ml; such as a particle density of at least 3.5 g/ml; e.g. a particle density of at least 4.0 g/ml; such as a particle density of at least 4.5 g/ml. Even more preferably, the adsorbent particle comprises a particle having a mean volume particle diameter of at the most 125 μm and a particle density of at least 1.5 g/ml; such as a particle density of at least 1.6 g/ml; e.g. a particle density of at least 1.9 g/ml; such as a particle density of at least 2.0 g/ml; e.g. a particle density of at least 2.3 g/ml; such as a particle density of at least 2.5 g/ml; e.g. a particle density of at least 2.8; e.g. a particle density of at least 3.0 g/ml; such as a particle density of at least 3.5 g/ml; e.g. a particle density of at least 4.0 g/ml; such as a particle density of at least 4.5 g/ml. Even more preferably, the adsorbent particle comprises a particle having a mean volume particle diameter of at the most 100 μm and a particle density of at least 1.5 g/ml; such as a particle density of at least 1.6 g/ml; e.g. a particle density of at least 1.9 g/ml; such as a particle density of at least 2.0 g/ml; e.g. a particle density of at least 2.3 g/ml; such as a particle density of at least 2.5 g/ml; e.g. a particle density of at least 2.8; e.g. a particle density of at least 3.0 g/ml; such as a particle density of at least 3.5 g/ml; e.g. a particle density of at least 4.0 g/ml; such as a particle density of at least 4.5 g/ml. Even more preferably, the adsorbent particle comprises a particle having a mean volume particle diameter of at the most 90 μm and a particle density of at least 1.5 g/ml; such as a particle density of at least 1.6 g/ml; e.g. a particle density of at least 1.9 g/ml; such as a particle density of at least 2.0 g/ml; e.g. a particle density of at least 2.3 g/ml; such as a particle density of at least 2.5 g/ml; e.g. a particle density of at least 2.8; e.g. a particle density of at least 3.0 g/ml; such as a particle density of at least 3.5 g/ml; e.g. a particle density of at least 4.0 g/ml; such as a particle density of at least 4.5 g/ml.

The adsorbent particle used according to the invention must be at least partly permeable to the biomolecular substance to be isolated in order to ensure a significant binding capacity in contrast to impermeable particles that can only bind the target molecule on its surface resulting in relatively low binding capacity. The adsorbent particle may be of an array of different structures, compositions and shapes.

The high density of the adsorbent particle is, to a great extent, achieved by inclusion in a porous polymer phase, of a certain proportion of a dense non-porous core material. The non-porous core preferably has a density of at least 4.0 g/mL, such as at least 5.0 g/mL, e.g. at least 8.0 g/mL, such as at least 10 g/mL, e.g. at least 15 g/mL. Typically, the non-porous core material has a density in the range of about 4.0-25 g/ml, such as about 4.0-20 g/ml, e.g. about 4.0-15 g/mL, such as 12-19 g/ml, e.g. 14-18 g/ml, such as about 6.0-15.0 g/mL, e.g. about 6.0-10 g/ml.

Other types of high density adsorbent particles are based on particles made out of a porous high density material, such as zirconium oxide, in which pores ligands for adsorption may be immobilised either directly to the high density material or to porous polymer networks filled into the pores of the high density material, see e.g. U.S. Pat. No. 6,036,861 and WO 99/51316. Although being an attractive way of providing a high density and small adsorbent particle such types of adsorbents will generally have some draw backs due to diffusion restriction in the porous structure and a high volume content of the high density phase resulting in low accessible protein binding volumes

It is of central importance to a broad range of preferred embodiments of the invention that the adsorbent particle employed according to the invention has a high accessible protein binding volume. In the present context the term “particle accessible protein binding volume” relates to the relative pore volume of any specific particle type and is expressed as volume percent relative to the volume of the entire bead (i.e. the volume occupied by pores/the total volume of the bead×100%). Thus if too much of the particle volume is occupied by the high density material only low column productivities can be achieved.

In an embodiment of the present invention the particle accessible protein binding volume of the adsorbent may be at least 20%, more preferably at least 30%, still more preferably at least 40%, still more preferably at least 50%, still more preferably at least 55%, still more preferably at least 60%, still more preferably at least 65%, still more preferably at least 70%, still more preferably at least 75%, still more preferably at least 80%, still more preferably at least 85% and still more preferably at least 90%.

In an embodiment of the present invention the adsorbent may have a dynamic binding capacity at 10% break-through for said at least one specific protein of at least 5 g per litre sedimented adsorbent, more preferably at least 10 g per litre, even more preferably at least 15 g per litre, still more preferably at least 20 g per litre, still more preferably at least 25 g per litre, still more preferably at least 30 g per litre, still more preferably at least 35 g per litre, still more preferably at least 40 g per litre, still more preferably at least 50 g/litre, still more preferably at least 60 g/litre.

When the protein solution is added to the adsorbent column the ratio between the adsorbent particle present in the column and the protein solution may be optimized in order to retain a high capacity of the adsorbent and to obtain a high purity of the protein or proteins to be isolated. In a preferred embodiment of the present invention the adsorbent present in the column relative to the protein solution to be loaded on to the column are provided at a ratio of at least 1:100, such as at least 1:50, e.g. at least 1:30, such as at least 1:15, e.g. 1:10, such as 1:5, such as 1:1, such as 1:0,5 measured on a volume/volume basis.

Thus, the adsorbent particles may be constituted by a number of chemically derivatised porous materials having the necessary density, diameter and/or binding capacity to operate at the given flow rates per se. The particles may for example be of the conglomerate type, as described in WO 92/00799, having at least two non-porous cores surrounded by a porous material, or of the pellicular type having a single non-porous core surrounded by a porous material. Another type of adsorbent particles may be constituted by a porous high density material (such as controlled pore glass or porous ceramics) having adsorbning ligands present in the pores of the material either covalently coupled to the porous material or coupled to a polymeric material filled into the pores.

In the present context the term “conglomerate type” relates to a particle of a particulate material, which comprises beads of core material of different types and sizes, held together by the polymeric base matrix, e.g. an core particle consisting of two or more high density particles held together by a surrounding polymeric base matrix (e.g. agarose).

In the present context the term “pellicular type” relates to a composite of particles, wherein each particle consists of only one high density core material coated with a layer of the porous polymeric base matrix, e.g. a high density stainless steel bead coated with agarose.

Accordingly the term “at least one high density non-porous core” relates to either a pellicular core, comprising a single high density non-porous particle or it relates to a conglomerate core comprising more that one high density non-porous particle.

The adsorbent particle, as stated, may comprise a high density non-porous core with a porous material surrounding the core, and said porous material optionally comprising a ligand at its outer surface.

In the present context the term “core” relates to the non-porous core particle or core particles which are present inside the adsorbent particle. The core particle or core particles may be incidental distributed within the porous material and is not limited to be located in the centre of the adsorbent particle.

The non-porous core constitutes typically of at most 70% of the total volume of the adsorbent particle, such as at most 60%, preferably at most 50%, preferably at most 40%, preferably at most 30%, preferably at most 20%, preferably at most 15%, preferably at most 10% preferably at most 5%.

Examples of suitable non-porous core materials are inorganic compounds, metals, heavy metals, elementary non-metals, metal oxides, non metal oxides, metal salts and metal alloys, etc. as long as the density criteria above are fulfilled. Examples of such core materials are metal silicates metal borosilicates; ceramics including titanium diboride, titanium carbide, zirconium diboride, zirconium carbide, tungsten carbide, silicon carbide, aluminum nitride, silicon nitride, titanium nitride, yttrium oxide, silicon metal powder, and molybdenum disilide; metal oxides and sulfides, including magnesium, aluminum, titanium, vanadium, chromium, zirconium, hafnium, manganese, iron, cobalt, nickel, copper and silver oxide; non-metal oxides; metal salts, including barium sulfate; metallic elements, including tungsten, zirconium, titanium, hafnium, vanadium, chromium, manganese, iron, cobalt, nickel, indium, copper, silver, gold, palladium, platinum, ruthenium, osmium, rhodium and iridium, and alloys of metallic elements, such as alloys formed between said metallic elements, e.g. stainless steel; crystalline and amorphous forms of carbon, including graphite, carbon black and charcoal. Preferred non-porous core materials are tungsten carbide, tungsten, steel and titanium beads such as stainless steel beads.

The porous material may be a polymeric base matrix used as a means for covering and keeping multiple (or a single) core materials together and within the adsorbent particle and as a means for binding the adsorbing ligand.

The polymeric base matrix may be sought among certain types of natural or synthetic organic polymers, typically selected from i) natural and synthetic polysaccharides and other carbohydrate based polymers, including agar, alginate, carrageenan, guar gum, gum arabic, gum ghatti, gum tragacanth, karaya gum, locust bean gum, xanthan gum, agaroses, celluloses, pectins, mucins, dextrans, starches, heparins, chitosans, hydroxy starches, hydroxypropyl starches, carboxymethyl starches, hydroxyethyl celluloses, hydroxypropyl celluloses, and carboxymethyl celluloses; ii) synthetic organic polymers and monomers resulting in polymers, including acrylic polymers, polyamides, polyimides, polyesters, polyethers, polymeric vinyl compounds, polyalkenes, and substituted derivatives thereof, as well as copolymers comprising more than one such polymer functionally, and substituted derivatives thereof; and iii) mixture thereof.

A preferred group of polymeric base matrices are polysaccharides such as agarose.

The investigators of the present invention have found that in order to ensure an efficient adsorption at high flow rates it may be necessary to minimise the mean volume particle diameter of the adsorbent particle. Thus, in a preferred embodiment of the present invention the adsorbent particle has a mean volume particle diameter of at the most 250 μm, typically a mean volume particle diameter in the range of about 30 μm to 200 μm. The adsorbent particle typically has a mean volume particle diameter of at most 175 μm, particularly at most 150 μm, more preferably at most 125 μm, 100 μm or 90 μm, more preferably at 80 μm and most preferably at most 70 μm.

From a productivity point of view it is important that the adsorbent is able to bind a high amount of the biomolecular substance per volume unit of the adsorbent. Thus we have found that it is preferable to apply adsorbents having a polymeric phase (i.e. the permeable polymeric network where a ligand is positioned and whereto the actual adsorption is taking place) which constitutes at least 50% of the adsorbent particle volume, preferably at least 70%, more preferably at least 80% and most preferably at least 90% of the volume of the adsorbent particles.

The Ligand

The isolation process of the one or more protein(s) may be provided and facilitated by attaching a suitable ligand to the adsorbent. In an embodiment of the present invention the adsorbent comprises a functionalised matrix polymer carrying a plurality of ligands comprising covalently attached functional groups. Any type of ligand or adsorption principle that will lead to the binding of the proteins and peptides according to the invention may be employed.

In a preferred embodiment of the present invention the ligand comprises an aromatic or heteroaromatic ring-system and one or more acidic groups.

In the present context the term “functionalised matrix polymer” relates to the anchoring site for the ligand promoting the desired protein adsorption characteristics. Depending on the adsorbent particle structure the matrix polymer may form the backbone or skeleton defining the physical shape of the adsorbent particle or it may be a polymer that is occupying the pores of another material that serve as the particle backbone or skeleton. In preferred embodiments the functionalised matrix polymer is a synthetic or natural organic polymer, such as a polysaccharide (e.g. poly-acrylic polymers, agarose or cellulose), or it may be an inorganic polymer, such as silica. In special cases the matrix polymer itself may constitute the protein adsorption site in which case it in not necessary to immobilise further ligands onto the polymer.

In an embodiment of the present invention the adsorbent comprises a functionalised matrix polymer carrying a plurality of covalently attached functional groups, said groups having the general formula:

M-SP1-X-Alk,

wherein M designates the adsorbent polymer; SP1 designates an optional spacer optionally substituted with -A-SP2-ACID, -A, or -ACID; X designates —O—, —S—, —NH—, or —NAlk-; Alk may be absent, -A-SP2-ACID, -A, -ACID or C₁₋₄ alkyl, where C₁₋₄ alkyl may be optionally substituted with -A-SP2-ACID, -A, or -ACID; A designates an optionally substituted aromatic or heteroaromatic moiety; SP2 designates an optional spacer; and ACID designates one or more acidic groups; wherein at least one of SP1 or Alk is substituted with -A-SP2-ACID or -A, and at least one of SP1 or Alk comprise -ACID and wherein at least one of SP1 or Alk is present. If Alk is absent, X will also be absent.

In an embodiment of the present invention the adsorbent may be coupled with a ligand carrying a positive charge at pH value at pH 10 or lower, such as pH 9 or lower, e.g. pH 8 or lower, such as pH 7 or lower, e.g. pH 6 or lower, such as pH 5 or lower, e.g. pH 4 or lower.

Further is has been found that the functional groups should not be too large in size and complexity in order to obtain a high binding capacity and a high chemical stability of the adsorbent. Thus it has been found that a larger size in terms of molecular weight and number of ring-systems present in the functional group in many instances only increase the cost of the adsorbent without giving the benefit of a higher binding capacity in terms of the amount of protein that can be bound per litre adsorbent. Also the molar concentration of the covalently attached functional group achievable on the adsorbent may be lower if a large molecular size of the functional group is employed (presumably due to steric hindrance).

Thus, in an embodiment of the invention the covalently attached functional groups comprise a maximum of three mono- or bicyclic aromatic or heteroaromatic ring-systems for each functional group attached to the matrix polymer, more preferably a maximum of two mono- or bicyclic aromatic or heteroaromatic ring-systems and even more preferably a maximum of one mono- or bicyclic aromatic or heteroaromatic ring-systems for each functional group attached to the matrix polymer. Likewise, in an embodiment of the invention the covalently attached functional groups comprise a maximum of three acidic groups, preferably a maximum of 2 acidic groups and most preferably a maximum of one acidic group attached to each aromatic or heteroaromatic ring-system present in the covalently attached functional groups.

In a preferred embodiment of the invention the one or more acidic groups are chosen from the group of carboxylic acids, sulfonic acids, phosphonic acids, boronic acids and combinations hereof.

In an embodiment of the present invention the ligand may be derived from a diethylaminoethyl group, a polyalkylene imine, an alkyl-amine, an alkyl-diamine or a polyallylamine. Preferably, alkyl-amine or alkyl-diamine having a chain-length of 3-14 atoms and 1-5 functional amine groups may be suitable. Atoms to form part of the chain may involve C (carbon), N (nitrogen), O (oxygen) and/or S (sulfur).

In yet an embodiment of the present invention the adsorbent may comprise a ligand, having both aromatic groups and amino groups such as an aromatic amine or an aromatic diamine. Preferably, the aromatic diamine is 1,4-xylene-diamine or isomers of 1,4-xylene-diamine.

In yet an embodiment of the present invention the adsorbent may be coupled with a ligand having an acid group, an aromatic or heteroaromatic moiety, a bicyclic substituted heteroaromatic group or any combination hereof, such as a ligand having an acid group and an aromatic or heteroaromatic moiety, a ligand having an acid group and a bicyclic substituted heteroaromatic group or an aromatic or heteroaromatic moiety and a bicyclic substituted heteroaromatic group.

In another embodiment of the present invention the ligand comprises a bicyclic substituted heteroaromatic group which may be derived from compounds selected from the group consisting of benzimidazoles, benzothiazoles, and benzoxazoles.

In an embodiment of the present invention the ligand may be an aromatic or heteroaromatic acid selected from the group consisting of carboxylic acids, sulfonic acids, phosphonic acids, and boronic acids. Preferably, the ligand may be selected from the group consisting of 2-mercaptobenzoic acid, 2-mercaptonicotinic acid, 2-aminobenzoic acid, 3-aminobenzoic acid, and 4-aminobenzoic acid, 4-hydroxyphenyl-mercapto-acetic acid, 4-hydroxyphenyl-mercapto-propionic acid, 4-hydroxyphenyl-mercapto-butanoic acid, 2,3-dihydroxy-benzoic acid, 2,4 dihydroxy-benzoic acid, 2,5 di-hydroxy-benzoic acid, 2,6 dihydroxy-benzoic acid, 3,4-dihydroxy-benzoic acid, 3,5-dihydroxy-benzoic acid, mercaptobenzimidazole sulfonic acid, orthanilic acid, metanilic acid, sulphanilic acid, 4-methylaniline-2-sulphonic acid, 4-methoxyaniline-2-sulphonic acid, aniline-2,5-disulphonic acid, N-methylmetanilic acid, 7-amino-1-naphthol-3-sulphonic acid, 1-naphthol-4-sulphonic acid, 2-naphthol-6-sulphonic acid and 2-hydroxy-3-naphthoic acid, and 2-mercaptobenzimidazole-sulphonic acid.

In an embodiment of the present invention the ligand may be an N-benzoyl amino acid or an N-benzoyl amino acid comprising thiol or mercapto groups.

In yet an embodiment of the present invention the ligand may be coupled to the adsorbent through a thio-ether linkage, an amine linkage, or an oxygen-ether linkage.

The optimal concentration of the covalently attached functional groups (the ligands) on the polymeric adsorbent backbone (also frequently referred to as the density of functional groups or the ligand concentration) will depend on the detailed structure of the functional group and the type of adsorbent material used to prepare the adsorbent.

In order to ensure an optimal adsorption strength and productivity of the adsorbent it has been found that the ligand concentration on the adsorbent may be significant. Thus, in a suitable embodiment, the adsorbent carries ligands for adsorption of the biomolecular substances in a concentration of at least 20 mM, such as at least 30 mM or at least 40 mM, preferably at least 50 mM and most preferably at least 60 mM.

However, generally it would be preferred that the adsorbent has a concentration of covalently attached functional groups in the range of 5-500 millimole per liter adsorbent in its sedimented (packed) bed state, more preferably in the range of 10-250 millimole per liter, still more preferably in the range of 10-125 millimole per liter, still more preferably in the range of 15-100 millimole per liter, still more preferably in the range of 20-80 millimole per liter still more preferably in the range of 25-75 millimole per liter still more preferably in the range of 30-60 millimole per liter.

In an embodiment of the present invention the covalently attached functional groups may be attached to the adsorbent by any type of covalent bond known per se to be applicable for this purpose, either by a direct chemical reaction between the ligand and the adsorbent or by a preceding activation of the adsorbent or of the ligand with a suitable reagent known per se making it possible to link the polymeric matrix backbone and the functional group. Examples of such suitable activating reagents are epichlorohydrin, epibromohydrin, allyl glycidylether; bis-epoxides such as butanedioldiglycidylether; halogen-substituted aliphatic compounds such as di-chloro-propanol, carbonyldiimidazole; aldehydes such as glutaric dialdehyde; quinones; periodates such as sodium-meta-periodate; carbodiimides; sulfonyl chlorides such as tosyl chlorides and tresyl chlorides; N-hydroxy succinimides; 2-fluoro-1-methylpyridinium toluene-4-sulfonates; oxazolones; maleimides; pyridyl disulfides; and hydrazides. Among these, the activating reagents leaving a spacer group SP1 different from a single bond, e.g. epichlorohydrin, epibromohydrin, allyl-glycidylether; bis-epoxides; halogen-substituted aliphatic compounds; aldehydes; quinones; cyanogen bromide; chloro-triazines; oxazolones; maleimides; pyridyl disulfides; and hydrazides, are preferred.

Especially interesting activating reagents are epoxy-compounds such as epichlorohydrin, allyl-glycidylether and butanedioldiglycidylether and polyglycidylethers such as glycerol polyglycidylether. In certain cases wherein the stability of the covalent binding of the functional group can be shown to be stable to treatment with sodium hydroxide e.g. 0.1 M to 2 M sodium hydroxide the activating reagent may be based on triazine derived reagents e.g. chloro-triazines such as cyanuric chloride.

The above mentioned possibilities makes it relevant to define the presence of an optional spacer SP1 lining the polymeric adsorbent backbone (also referred to as the matrix polymer) and the functional group. In the present context the spacer SP1 may be considered as being part of the activating reagent, which forms the link between the matrix polymer and the functional group. Thus, the spacer SP1 may corresponds to the activating reagents and the coupling reactions involved. In some cases, e.g. when using carbodiimides, the activating reagent forms an activated form of the matrix polymer or of the functional group reagent. After coupling no parts of the activating reagent is left between the functional group and the matrix polymer, and, thus, SP1 is simply a single bond.

In other cases the spacer SP1 may be an integral part of the functional group effecting the binding characteristics, i.e. the functional group, and this will be especially significant if the spacer SP1 comprises functionally active sites or substituents such as thiols, amines, acidic groups, sulfone groups, nitro groups, hydroxy groups, nitrile groups or other groups able to interact through hydrogen bonding, electrostatic bonding or repulsion, charge transfer or the like.

In still other cases the spacer SP1 may comprise an aromatic or heteroaromatic ring, which plays a significant role for the binding characteristics of the adsorbent. This would for example be the case if quinones or chlorotriazines where used as activation agents for the adsorbent or the functional group.

In a further case, the spacer SP1 may be a single bond or a biradical derived from an activating reagent selected from epichlorohydrin, allyl-glycidylether, allylbromide, bis-epoxides such as butanedioldiglycidylether, halogen-substituted aliphatic compounds such as 1,3-dichloropropan-2-ol, aldehydes such as glutaric dialdehyde, quinones, cyanogen bromide, chloro-triazines such as cyanuric chloride, 2-fluoro-1-methylpyridinium toluene-4-sulfonates, maleimides, oxazolones, and hydrazides.

In an embodiment of the present invention the spacer SP1 may be a short chain aliphatic biradical, e.g. having the formula: —CH₂—CH(OH)—CH₂— (derived from epichlorohydrin), —(CH₂)₃—O—CH₂—CH(OH)—CH₂— (derived from allyl-glycidylether) or —CH₂—CH(OH)—CH₂—O——(CH₂)₄—O—CH₂—CH(OH)—CH₂— (derived from butane-dioldiglycidylether; or a single bond.

In an embodiment of the present invention the adsorbents typically comprises a ligand comprising aromatic or heteroaromatic groups (radicals) selected from the groups comprising i) ligands comprising the following types as functional groups: benzoic acids such as 2-aminobenzoic acids, 3-aminobenzoic acids, 4-aminobenzoic acids, 2-mercaptobenzoic acids, 4-amino-2-chlorobenzoic acid, 2-amino-5-chlorobenzoic acid, 2-amino-4-chlorobenzoic acid, 4-aminosalicylic acids, 5-aminosalicylic acids, 3,4-diaminobenzoic acids, 3,5-diaminobenzoic acid, 5-aminoisophthalic acid, 4-aminophthalic acid; cinnamic acids such as hydroxy-cinnamic acids; nicotinic acids such as 2-mercaptonicotinic acids; naphthoic acids such as 2-hydroxy-l-naphthoic acid; quinolines such as 2-mercaptoquinoline; tetrazolacetic acids such as 5-mercapto-1-tetrazolacetic acid; thiadiazols such as 2-mercapto-5-methyl-1,3,4-thiadiazol; benzimidazols such as 2-amino-benzimidazol, 2-mercaptobenzimidazol, and 2-mercapto-5-nitrobenzimidazol; benzothiazols such as 2-aminobenzothiazol, 2-amino-6-nitrobenzothiazol, 2-mercaptobenzothiazol and 2-mercapto-6-ethoxybenzothiazol; benzoxazols such as 2-mercaptobenzoxazol; thiophenols such as thiophenol and 2-aminothiophenol; 2-(4-aminophenylthio)acetic acid; aromatic or heteroaromatic sulfonic acids and phosphonic acids, such as 1-amino-2-naphthol-4-sulfonic acid and phenols such as 2-amino-4-nitro-phenol. It should be noted that the case where M is agarose, SP1 is derived from vinyl sulfone, and L is 4-aminobenzoic acid may be specifically disclaimed in relation to the solid phase matrices according to the invention, cf. WO 92/16292, most preferably amino-benzoic acids like 2-amino-benzoic acid, 2-mercapto-benzoic acid, 3-aminobenzoic acid, 4-aminobenzoic acid, 4-amino-2-chlorobenzoic acid, 2-amino-5-chlorobenzoic acid, 2-amino-4-chlorobenzoic acid, 4-aminosalicylic acids, 5-aminosalicylic acids, 3,4-diaminobenzoic acids, 3,5-diaminobenzoic acid, 5-5-aminoisophthalic acid, 4-aminophthalic acid.

Generally, the coupling using divinyl sulphone may not be suitable because the divinyl sulphone coupling is unstabil when contacted with an alkaline and as alkalines are presently the most suitable and used cleaning agents, adsorbents coupled with divinyl sulphone are not being considered industrial relevant; ii) ligands comprising 2-hydroxy-cinnamic acids, 3-hydroxy-cinnamic acid and 4-hydroxy-cinnamic acid iii) ligands comprising a carboxylic acid and an amino group as substituents such as 2-amino-nicotinic acid, 2-mercapto-nicotinic acid, 6-amino-nicotinic acid and 2-amino-4-hydroxypyrimidine-carboxylic acid iv) ligand comprising radicals derived from a benzene ring fused with a heteroaromatic ring system, e.g. a ligand selected from benzimidazoles such as 2-mercapto-benzimidazol and 2-mercapto-5-nitro-benzimidazol; benzothiazols such as 2-amino-6-nitrobenzothiazol, 2-mercaptobenzothiazol and 2-mercapto-6-ethoxybenzothiazol; benzoxazols such as 2-mercaptobenzoxazol; and v) ligands chosen from the group of thiophenols such as thiophenol and 2-aminothiophenol.

Within the embodiment wherein the ligand is selected from group i)-v) mentioned above, the adsorbents typically have a dynamic binding capacity of at least 10 g of biomolecular substance per litre, more preferably at least 20 g per litre, still more preferable at least 30 g per litre when tested according to the process conditions used in the relevant application.

The binding capacity of the adsorbent may be determined in terms of its binding capacity to bovine serum albumin (BSA). The binding capacity is typically such that at least 10 g/L of BSA binds according to test Method A.

Method A

Method A is a method used for determination of the bovine albumin binding capacity of selected adsorbents consisting of the following process:

Bovine serum albumin solution pH 4.0 (BSA pH 4.0): Purified bovine serum albumin (A 7906, Sigma, USA) is dissolved to a final concentration of 2 mg/ml in 20 mM sodium citrate pH 4.0. Adsorbents are washed with 50 volumes of 20 mM sodium citrate pH 4.0 and drained on a suction filter.

A sample of 1.0 ml suction drained adsorbent is placed in a 50 ml test tube followed by the addition of 30 ml of BSA, pH 4.0.

The test tube is then closed with a stopper and the suspension incubated on a roller mixer for 2 hours at room temperature (20-25° C.). The test tube is then centrifuged for 5 min. at 2000 RPM in order to sediment the adsorbent completely. The supernatant is then isolated from the adsorbent by pipetting into a separate test tube, avoiding the carry-over of any adsorbent particles and filtered through a small non-adsorbing 0.2 μm filtre (Millipore, USA). Following this a determination of the concentration of non-bound BSA in the supernatant is performed by measuring the optical density (OD) at 280 nm on a spectrophotometer.

The amount of BSA bound to the adsorbent is then calculated according to the following formula:

mg BSA bound per ml suction drained adsorbent=(1-(OD of test supernatant/OD of BSA starting solution))×60 mg BSA/ml adsorbent.

The Column

The adsorbent capable of capturing the one or more protein(s) may be held within a column or it may not be held within a column. In the present context the term “column” relates to any kind of container which can be supplied with at least one inlet and at least one outlet for the application of the protein solution to the column and subsequent to elute the protein. The inlet and the outlet may for certain columns be the same (e.g. for batch adsorption tanks). The column may be in the form of an Expanded bed adsorption (EBA) column, packed bed column, a fluidized bed adsorption column, a suspended bed adsorption column, membrane reactor, or a batch adsorption tank. The adsorbent column may be used in either a batch system or in a continuous system. Typically packed bed columns and expanded bed adsorption columns operate under plug flow conditions (i.e. no liquid back-mixing and turbulence in the adsorbent bed), while suspended bed columns and batch adsorption tanks operate with a high degree of mixing at least in the major part of the column volume.

The fact that the EBA technology generally can work efficiently with non-clarified protein solution makes it attractive for the isolation of proteins. Compared to processes based on packed bed adsorption techniques EBA may offer a robust process comprising fewer steps and thus result in increased yields and an improved process economy. Due to the expansion of the adsorbent bed during execution of an EBA process, EBA columns may further be scaled up to industrial scale without any significant considerations regarding increased back pressures or breakdown of the process due to clogging of the system which often is a problem when using packed bed columns.

In accordance with the present invention the protein solution is applied to a packed bed column or an expanded bed column comprising an adsorbent.

In the present context the term “packed bed” relates to embodiments wherein the adsorbent particles are employed in columns operating with the particles in a sedimented or packed state wherein all particles are fixed on top of each other. Often packed bed columns are equipped with top and bottom adaptors defining and fixing the whole adsorbent bed to avoid any movement of the particle during operation.

In the present context the term “expanded bed” relates to embodiments wherein the adsorbent particles are employed in columns allowing the adsorbent to expand with an upward liquid flow through the column. The column will be designed to avoid excessive liquid mixing and turbulence in the column while the individual adsorbent particles are kept in a non-fixed, dynamic state moving only in a narrow local zone in the column. While preferred expanded beds have a small mixing zone in the bottom part of the column where incoming liquid is distributed throughout the cross-section of the column, expanded beds generally operate under plug flow conditions in similarity with packed beds. In an embodiment of the present invention the adsorbent is held in an Expanded bed adsorption column and preferably used for the large-scale isolation of one or more protein(s) from a protein solution.

In the case where the adsorbent is not held within a column it may be a solid phase, such as for membrane based adsorption, e.g. a membrane filter, fibers or sheets, whereto the ligand is coupled.

Whenever the adsorbent is in the form of permeable or semi-permeable membranes, fibres or sheets the contacting between the adsorbent and the protein solution may generally be performed by pumping/forcing the protein solution across the surface and/or through a porous structure of the membrane or sheet to ensure that the one or more plasma or serum protein may be coming in close contact with the covalently attached functional groups on the surface and/or in the adsorbents.

The process Parameters

As mentioned above the protein solution comprising the protein(s) or peptides of interest may be adjusted to having a preset pH and a preset ionic strength or conductivity.

In the present context the term “preset” relates to the adjustment of the pH, ionic strength or conductivity, respectively, to a specific and predetermined value for the purpose of selecting the ability of the adsorbent for binding the one or more protein(s) of interest and thereby increasing the efficiency of the adsorbent for protein(s) isolation.

The ionic strength and conductivity of the protein solutions according to the present invention are related entities in that both entities are functions of the concentration of ions in the solution. There is, however, no direct theoretical correspondence between them.

When considering an ion-containing solution, it is relatively easy for the person skilled in the art to calculate the amount of e.g. an inorganic salt necessary to achieve a certain ionic strength. Conversely, when the person skilled in the art is faced with the problem of determining the ionic strength without knowing the amount of added salt, it is difficult to make an accurate assessment since ionic strength is a theoretical entity calculated from both the concentration of ions and the charge of the ions. In this situation it is considerably easier for the person skilled in the art to measure the conductivity. For these reasons, the terms “ionic strength” and “conductivity” are used in the present context to characterise the same conditions. When referring to preferred ranges for these two entities, though, it is not meant to say that there is any correspondence between the indicated lower or upper limits of the ionic strength and conductivity, respectively.

For obtaining the protein(s) or peptides from the adsorbent the protein(s) or peptides may be eluted with one or more buffer(s). Optionally, adsorbent may be washed with a washing buffer before being subjected to the elution buffer. In an embodiment of the present invention the adsorbent is washed with a washing buffer to wash out non-bound material before eluting one or more protein(s) from the adsorbent.

### Further Embodiments

In an embodiment of the present invention the method further involves the step of subjecting the adsorbent to an elution buffer (a first elution buffer) to elute at least one of said one or more protein(s) followed by subjecting the adsorbent to a second elution buffer to eluate further proteins or peptides. In a preferred embodiment Patatin is eluted from the adsorbent first followed by elution of the protease inhibitors.

For the purpose of removing unbound material, including unbound protein(s), the adsorbent may be subjected to a washing buffer. In an embodiment of the present invention the one or more protein(s) to be isolated from the protein solution may be washed out of the adsorbent with the washing buffer. This washing may be performed before subjecting the adsorbent to an elution buffer as described above.

In an embodiment washing and/or elution may be performed with a washing buffer and/or an elution buffer having a higher pH and/or a higher ionic strength than the preset pH and preset ionic strength of the protein solution.

In still another embodiment the washing buffer and/or the elution buffer may comprise one or more compounds having a hydrophobic as well as a negatively charged group within the same molecule e.g. negatively charged detergent such as octyl sulfate, bromphenol blue, octane sulfonate, sodium laurylsarcosinate, hexane sulfonate, sodium dodecyl sulfate, sodium caprylate.

For the purpose of obtaining one or more additional protein(s) from the adsorbent the process according to the present invention further comprises the step of eluting with one or more additional elution buffer(s) to elute remaining protein(s).

In the present context the term “additional elution buffer(s)” relates to the buffer(s) subsequently used for the elution of one or more protein(s), which remains bound to the adsorbent after the elution with the first elution buffer.

In the present context the term “remaining protein(s)” relates to the one or more protein(s) which remains bound to the adsorbent after being subjected to a first elution buffer and which protein(s) may subsequently be eluted by the addition of an additional elution buffer.

In an embodiment of the present invention the adsorbent may be washed with an additional washing buffer between each elution step.

In the present context the term “protein fraction” relates to the collections obtained from the adsorbent wherein the one or more protein(s) to be isolated may be located. This protein fraction may be subjected to further downstream processing for further isolation of the one or more protein(s) present. The further downstream processing may involve operations like filtration, centrifugation, sedimentation, microfiltration, precipitation and chromatography. In an embodiment of the present invention chromatography involves ion exchange chromatography, gel filtration, affinity chromatography, hydrophobic interaction chromatography and reversed phase chromatography, where the protein(s) may be bound to a second adsorbent in subsequent down stream processing.

In an embodiment of the present invention the further downstream processing may comprise the adsorption of the protein in the protein fraction(s) to an alkyl-amine such as an alkyl-diamine such as diamino-hexane, diamino-heptane, diamino-octane, diamino-nonane, diamino-decane and isomers or derivatives hereof.

It is an embodiment of the present invention to provide a process wherein multiple protein fractions are provided by each adsorption cycle such as at least 2 protein fractions, e.g. at least 3 protein fractions, such as at least 4 protein fractions, e.g. at least 5 protein fractions, such as at least 6 protein fractions. Preferably each of these protein fractions comprises a high yield of individual proteins without significant cross-contamination of the protein fraction(s) between the at least 2 proteins, such as at least 3 proteins e.g. at least 4 proteins, such as at least 5 proteins e.g. at least 6 proteins within the same protein fraction. In an embodiment of the present invention the amount of cross contamination in a protein fraction is less than 20%, such as less than 15%, e.g. less than 10%, such as less than 5%, e.g. less than 3%, such as less than 1%, e.g. less than 0.5%, such as less than 0.1%, e.g. less than 0.01%.

In the present context the term “cross-contamination” relates to the amount or content of protein not of interest which is present in the protein fraction. In some cases it is of interest to elute two or more proteins simultaneously in one elution circle and in this case the proteins intentionally eluted together are not considered contaminating. In an embodiment of the present invention the degree of cross-contamination of the individual protein in the protein fraction is at the most 20%, such as at the most 15%, e.g. at the most 10%, such as at the most 5%, e.g. at the most 3%, such as at the most 1%, e.g. at the most 0.5%, such as at the most 0.1%, e.g. at the most 0.01%.

The invention will be further illustrated in the following non-limiting figures and examples.

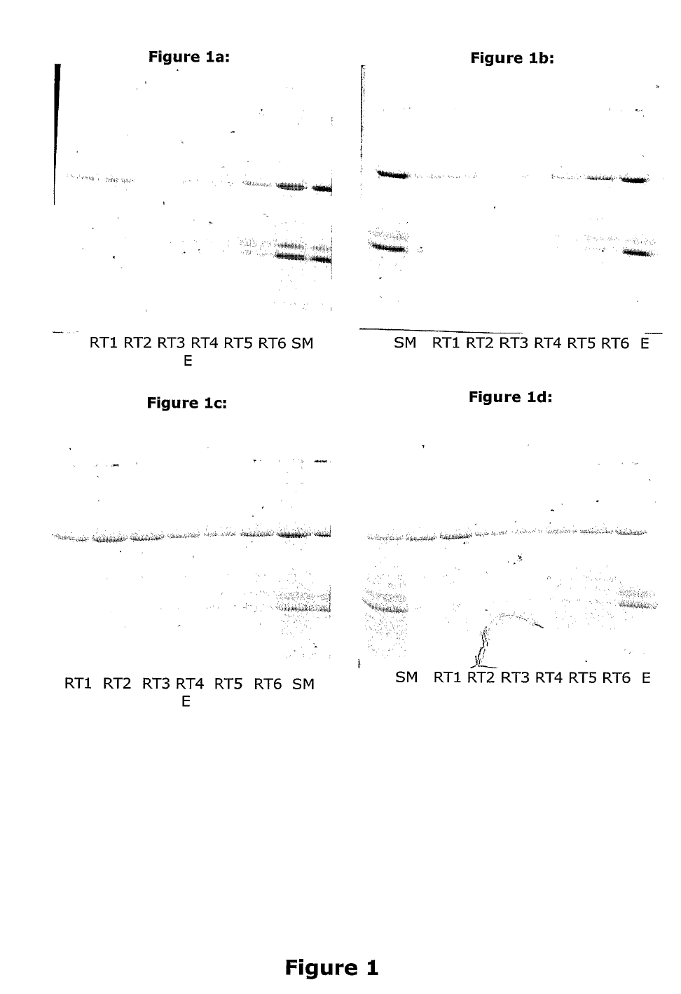

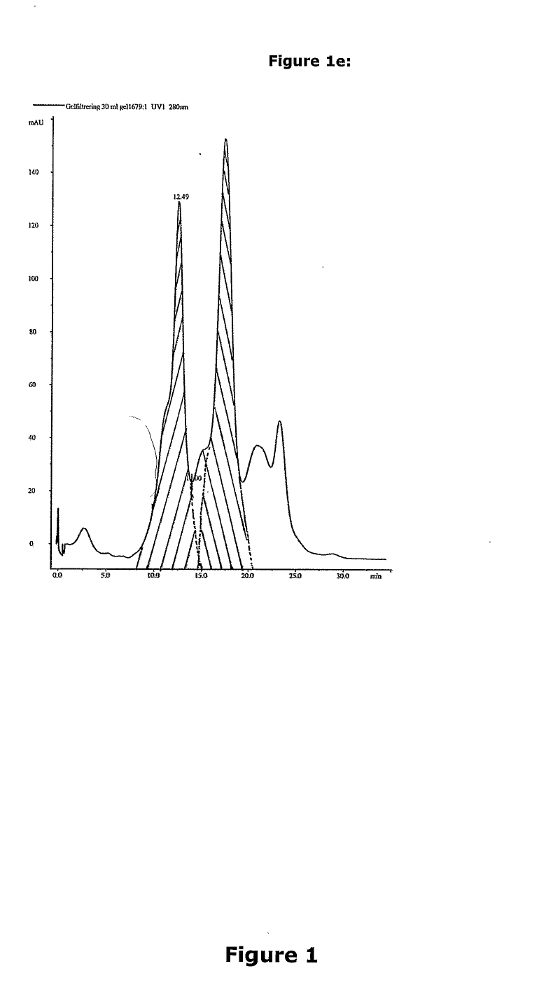

FIG. 1a: shows the content of protein by SDS-PAGE. Juice loaded at pH 4.0

FIG. 1b: shows the content of protein by SDS-PAGE. Juice loaded at pH 4.25

FIG. 1c: shows the content of protein by SDS-PAGE. Juice loaded at pH 4.5

FIG. 1d: shows the content of protein by SDS-PAGE. Juice loaded at pH 5.0

FIG. 1e: shows the calculation of peak area using a gel filtration curve

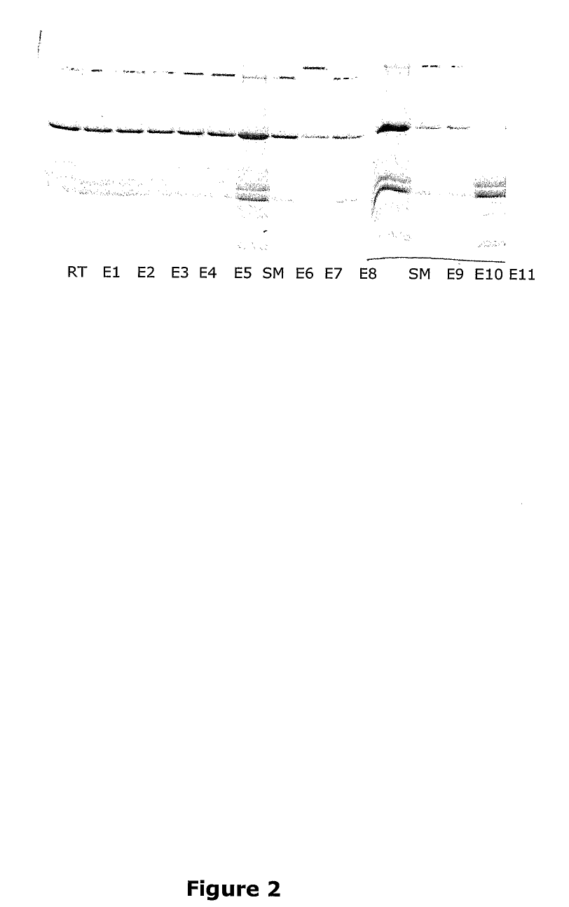

FIG. 2: shows the content of protein by SDS-PAGE. Juice flow rate was 20 cm/min

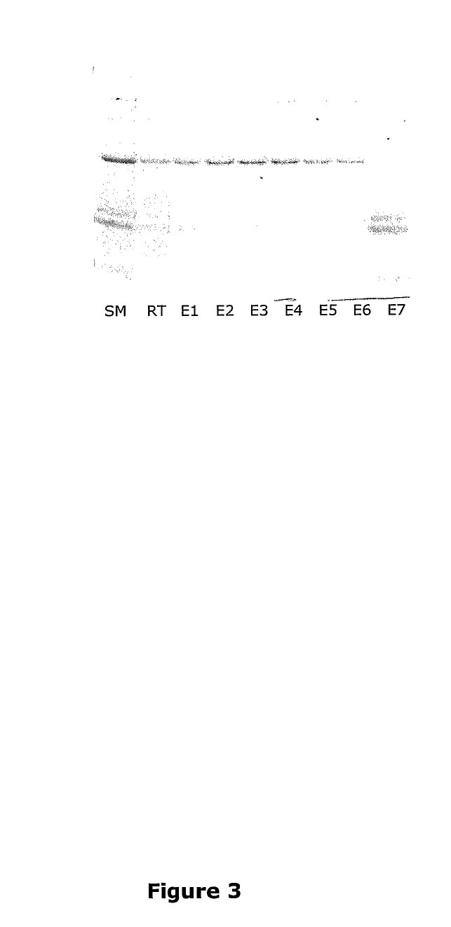

FIG. 3: shows the content of protein by SDS-PAGE. Juice flow rate was 30 cm/min

### EXAMPLES

Abbreviations

ABS Amino benzoic acid

CM Carboxy methyl

E Eluate

EBA Expanded bed chromatography

EU OD 280×volume of eluate

HBS Hydroxy benzoic acid

MBS Mercapto benzoic acid

MNS Mercapto nicotinic acid

NaCl Sodium citrate

OD Optical density

PI Protease inhibitor

RT Run through

SDS-PAGE Sodium dodecyl sulphate—poly acrylamide gel electrophorese

SM Start material

### Example 1

### Isolation of Potato Proteins from Potato Juice

Isolation of Patatin and protease inhibitors (PI) with expanded bed adsorption chromatography at 25° C.:

The potato juice was obtained by washing approximately 2 kilos of potatoes with water and thereafter soaking them in a cold and freshly made solution of 0.05% sodium pyrosulphite blowed through with nitrogen gas for 15 min. The solution comprising potatoes is transferred to a blender and blended until the potatoes are mashed. While blending there was added 100 ml of 0.05% sodium pyrosulphite per kilo of potatoes. The potato juice was collected by filtration under vacuum through two layers of paper. The filter cake was washed with 400 ml of 0.05% cold sodium pyrosulphite. In total 500 ml of 0.05% sodium pyrosulphite was used per kilo of potato.

Adsorbent

The adsorbent was based on agarose with integrated tungsten carbide particles resulting in a high density matrix of approximately 2.8 g/ml. The particle size was in the range of 40-200 μm with a mean volume diameter of 150 micron. The adsorbent comprises varying ligands that generally binds proteins in the pH range of 4-6.

Pre-Treatment of the Potato Juice

The pH in the juice was adjusted to different values in the range of 4-6 with 1 M hydrochloric acid in different experiments.

The experiment was performed in a FastLine® 10 expanded bed column (Ø=1 cm), UpFront Chromatography A/S, Copenhagen, Denmark. The column was packed with 50 cm of adsorbent (39.2 ml) and equilibrated with demineralised water, at 25° C.

The potato juice at different pH values was loaded onto the column with a linear flow rate of 15 cm/min. 300 ml was loaded and elution was performed with 20 mM NaOH.

Results

The concentration of protein in the eluates was estimated spectrophotometrically at 280 nm and expressed as EU adsorbed and subsequently obtained in the eluate per ml adsorbent in the column.

| | pH- | | | | | |
|---|---|---|---|---|---|---|
| | value during | | | EU/ml adsorbent | | |
| Ligand | adsorption | | | obtained in the eluate | | |
| Benzylamine | 6.5 | | | 59.6 | | |
| CM | 4.5 | | | 19.6 | | |
| 2-MNS | 4.5 | | | 62 | | |
| 4-ABS | 4.5 | 4.75 | 5.0 | 36.5 | 40 | 38 |
| 2,4-di ABS 4% (44817-wv) | 4.5 | | | 22.2 | | |
| 2-(4-aminophenylthio)acetic | 4.5 | | | 18.4 | | |
| 4-ABS | | 4.75 | | | 45 | |
| | | 5.3 | | | 41.5 | |
| | | 5.5 | | | 41 | |
| 2-MBS 6% | 5.0 | | | 43 | | |
| 3-MBS 6% | 5.0 | | | 45 | | |
| 4-MBS 6% | 5.0 | | | 39 | | |

EU: OD 280 × volume of eluate

### Example 2

### Isolation of Potato Proteins from Potato Juice Using 4-Amino Benzoic Acid as the Ligand

Adsorption of Patatin and protease inhibitors (PI) with expanded bed adsorption chromatography at 25° C.

The potato juice was produced according to the procedure described in example 1.

Adsorbent

Cat. No.: FastLine X051201, UpFront Chromatography A/S. The adsorbent is based on agarose with integrated tungsten carbide particles resulting in a density of approx. 2.8 g/ml. The particle size is in the range of 40-200 μm. The adsorbent comprises 4-amino benzoic acid as the ligand. The ligand concentration was approx. 50 micromoles per ml wet sedimented adsorbent.

Pre-Treatment of the Potato Juice

The pH in the extract was adjusted to different values in the range of 4-5 with 1 M hydrochloric acid in four independent experiments.

The experiment was performed in a FastLine® 10 expanded bed column (Ø=1 cm), UpFront Chromatography. The column was packed with 50 cm of adsorbent (39.2 ml) and equilibrated with 20 mM NaOH, 25° C.

The potato juice at different pH values was loaded onto the column with a linear flow rate of 15 cm/min. 300 ml was loaded. The run-through was collected in fractions of 50 ml.

The bound proteins were eluted from the adsorbent with 250 ml of 20 mM sodium hydroxide depending on the extract pH value.

The content of protein in each fraction was determined by SDS-PAGE (non-reduced 4-20% tris-glycine gradient gel, Coomassie stained, Anamed Elektrophorese GmbH cat.no.: TG42010, GF10002A+B). See FIG. 1a-1d.

Results

The eluate was analysed by gel filtration and the total protein concentration was estimated spectrophotometrically at 280 nm.

| | | Area | |
|---|---|---|---|
| Extract | OD 280 nm | patatin | Area PI |
| pH | of eluate | mAU * min | mAU * min |
| 4.0 | 5.5 | 165 | 223 |
| 4.25 | 6.9 | 212 | 290 |
| 4.5 | 7.5 | 226 | 334 |
| 5.0 | 8.9 | 216 | 378 |

Area of a protein is the area under the curve in a gel filtration (see Error! Reference source not found.e).

### Example 3

### Isolation of Potato Proteins from the Potato Juice at High Flow Rate

Isolation of the patatin and protease inhibitors (PI) with expanded bed adsorption chromatography at 25° C. To achieve a more cost efficient production method of potato juice proteins it is important to run with a high linear flow rate. The experiment is performed at a linear flow rate of 20 cm/min.

The potato juice and the adsorbent where the same as described in example 2.

Pre-Treatment of the Potato Juice

The pH in the extract was adjusted to 5.0 with 1 M hydrochloric acid at 25°. The experiment was performed in a FastLine® 10 expanded bed column (Ø=1 cm), UpFront Chromatography. The column was packed with 50 cm of adsorbent (39,2 ml) and equilibrated with 50 mM sodium acetate pH 5.0, 25° C.

The potato juice at pH 5.0 was loaded onto the column with a linear flow rate of 20 cm/min. 300 ml was loaded. The run-through was collected in fractions of 25 ml.

The bound proteins were eluted from the adsorbent with 300 ml of 20 mM sodium hydroxide.

The content of protein in each fraction was determined by SDS-PAGE (non-reduced 4-20% tris-glycine gradient gel, Coomassie stained, Anamed Elektrophorese GmbH cat.no.: TG42010, GF10002A+B). See FIG. 2.

Results

The eluate was analysed by gel filtration and the total protein concentration was estimated spectrophotometrically at 280 nm.

| | | Area | |
|---|---|---|---|
| Extract | | patatin | Area PI |
| pH | OD 280 nm | mAU * min | mAU * min |
| 5.0 | 5.3 | 377 | 821 |

Area of a protein is the area under the curve in a gel filtration

### Example 4

### Isolation of Potato Proteins from the Potato Juice at High Linear Flow Rate and High Bed Height

Isolation of Patatin and protease inhibitors (PI) with expanded bed adsorption chromatography at 25° C. To achieve a cost efficient production method of potato juice proteins it is important to run with a high linear flow rate. The experiment is performed at 30 cm/min.

The potato juice and the adsorbent where the same as described in example 2.

Pre-Treatment of the Potato Juice

The pH in the extract was adjusted to 5.0 with 1 M hydrochloric acid and heated to 25°.

The experiment was performed in a FastLine® 10 expanded bed column (Ø=1 cm), UpFront Chromatography. The column was packed with 50 cm of adsorbent (39.2 ml) and equilibrated with 50 mM sodium acetate pH 5.0, 25° C.

The potato juice at pH 5.0 was loaded onto the column with a linear flow rate of 30 cm/min. 300 ml was loaded. The run-through was collected in fractions of 50 ml.

The bound proteins were eluted from the adsorbent with 300 ml of 20 mM sodium hydroxide.

The content of protein in each fraction was determined by SDS-PAGE (non-reduced 4-20% tris-glycine gradient gel, Coomassie stained, Anamed Elektrophorese GmbH cat.no.: TG42010, GF10002A+B). See FIG. 3.

Results

The eluate was analysed by gel filtration and the total protein concentration was estimated spectrophotometrically at 280 nm.

| | | Area | | |
|---|---|---|---|---|
| Extract | | patatin | Area PI | |
| pH | OD 280 nm | mAU * min | mAU * min | Purity |
| 5.0 | 4.9 | 265 | 665 | 88% |

Area of a protein is the area under the curve in a gel filtration

### Example 5

### Isolation of Potato Proteins from Potato Juice using Different Elution Conditions

Isolation of patatin and protease inhibitors (PI) with expanded bed adsorption chromatography at 25° C. To investigate an alternative elution a constant amount of sodium chloride was added to different concentrations of sodium hydroxide. The experiments are performed with 0.15 M NaCl plus 5, 10, or 20 mM NaOH.

The potato juice and the adsorbent where the same as described in example 2.

Pre-Treatment of the Potato Juice

The pH in the extract was adjusted to 5.0 with 1 M hydrochloric acid and heated to 25°.

The experiment was performed in a FastLine® 10 expanded bed column (Ø=1 cm), UpFront Chromatography. The column was packed with 50 cm of adsorbent (39,2 ml) and equilibrated with 50 mM sodium acetate pH 5.0, 25° C.

The potato juice at pH 5.0 was loaded onto the column with a linear flow rate of 20 cm/min. 300 ml was loaded. The run-through was collected in fractions of 25 ml.

Results

The bound proteins were eluted from the adsorbent with 300, 250, and 175 ml of elution buffer respectively.

The eluate was analysed by gel filtration and the total protein concentration was estimated spectrophotornetrically at 280 nm.

| | | | Area | | |
|---|---|---|---|---|---|
| Extract | NaOH | | patatin | Area PI | |
| pH | Conc./mM | OD 280 nm | mAU * min | mAU * min | Purity |
| 5.0 | 5 | 5.3 | 107 | 214 | 80% |
| 5.0 | 10 | 6.2 | 120 | 322 | 93% |
| 5.0 | 20 | 7.4 | 166 | 370 | 92% |

Area of a protein is the area under the curve in a gel filtration

The adsorbent binds and eluates equal amounts of the potato juice proteins in each experiment. However, a 5 mM NaOH elution buffer results in larger elution volumes than 10 and 20 mM concentrations.

### REFERENCES

U.S. Pat. No. 5,187,154

U.S. Pat. No. 4,491,578

U.S. Pat. No. 4,906,457

U.S. Pat. No. 6,042,872

WO 99/51316

U.S. Pat. No. 6,036,861

WO 92/00799

WO 92/16292

1. A process for the isolation of one or more protein(s) or peptides from a protein solution wherein the protein is obtained from a potato derived source, said process comprising the steps of:
a) optionally adjusting the pH of the protein solution to a preset pH;
b) optionally adjusting the ionic strength or conductivity of the protein solution to a preset ionic strength or a preset conductivity;
c) applying said protein solution to an adsorption column comprising an adsorbent, wherein the adsorbent comprises a functionalized matrix polymer carrying a plurality of covalently attached functional groups comprising an aromatic or heteroaromatic ring-system and one or more acidic groups
d) optionally washing the column;
e) obtaining the one or more protein(s) from the adsorbent.

2. A process for the isolation of one or more protein(s) from a protein solution wherein the protein is selected from the group consisting of Patatin(s) and vegetable derived protease inhibitors, said process comprising the steps of:
a) optionally adjusting the pH of the protein solution to a preset pH;
b) optionally adjusting the ionic strength or conductivity of the protein solution to a preset ionic strength or a preset conductivity;
c) applying said protein solution to an adsorption column comprising an adsorbent, wherein the adsorbent comprises a functionalized matrix polymer carrying a plurality of covalently attached functional groups comprising an aromatic or heteroaromatic ring-system and one or more acidic groups;
d) optionally washing the column;
e) obtaining the one or more protein(s) from the adsorbent.

3. A process for the isolation of one or more protein(s) from a protein solution wherein the protein is selected from the group consisting of Patatin(s) and vegetable derived protease inhibitors, said process comprising the steps of:
a) optionally adjusting the pH of the protein solution to a preset pH;
b) optionally adjusting the ionic strength or conductivity of the protein solution to a preset ionic strength or a preset conductivity;
c) applying said protein solution to an adsorption column comprising an adsorbent, wherein the adsorbent has a density above 1.2 g/ml;
d) optionally washing the column;
e) obtaining the one or more protein(s) from the adsorbent.
